(19) United States
(12) Patent Application Publication
(10) Pub. No.: US 2019/0117616 A1
(43) Pub. Date: Apr. 25, 2019

(54) METHOD FOR REGULATING KDM4A, KDM4B, AND KDM4C ACTIVITY

(71) Applicant: National Tsing Hua University, Hsinchu City (TW)

(72) Inventors: Wen-Ching Wang, Hsinchu City (TW); Hsing-Jien Kung, Hsinchu City (TW)

(73) Assignee: National Tsing Hua University, Hsinchu City (TW)

(21) Appl. No.: 15/789,157

(22) Filed: Oct. 20, 2017

(51) Int. Cl. A61K 31/352; A61K 9/127

(52) U.S. Cl. CPC A61K 31/352; A61K 9/127

(57) ABSTRACT

The present invention relates to a method for inhibiting activity of a protein selected from the group consisting of KDM4A, KDM4B, and KDM4C, comprising the step of exposing the protein to an effective inhibiting amount of a compound having the formula

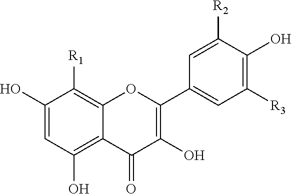

wherein R₁ is H or C₇O₅H₈; R₂ is H, OH, or OCH₃; and R₃ is H or OH.

The present invention also relates to a method for treating or lessening the severity of a disease, condition, or disorder in which modulation of KDM4A, KDM4B, or KDM4C is beneficial, which method comprises administering to a subject suffering from said disease, condition, or disorder a therapeutically-effective amount of:
a) a compound having the formula

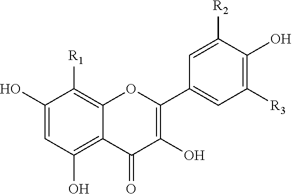

wherein R₁ is H or C₇O₅H₈; R₂ is H, OH, or OCH₃; and R₃ is H or OH; or
b) a composition comprising the compound and a pharmaceutically acceptable adjuvant, vehicle, or carrier.

### FIELD OF THE INVENTION

The present invention relates to the use of nature compounds to regulate processes or treat disorders that are modulated by KDM4A, KDM4B, or KDM4C or are caused by abnormal actions of KDM4A, KDM4B, or KDM4C in cells or organs of animals, humans, plants, or microorganisms. This invention relates to the use of nature compounds and their analogues or derivatives as KDM4A/KDM4B/KDM4C inhibitors and as therapeutic agents.

### BACKGROUND OF THE INVENTION

Prostate cancer is the most common cancer in the prostate gland of aged male population. It is now the six leading cause of cancer-related death in men and is the first in the United Kingdom and second in the United States. The standard prostate cancer therapies include surgery or hormonal castration therapy, referred as androgen deprivation therapy. Anti-androgens such as enzalutamide and inhibitors of anti-androgen synthesis such as abiraterone (inhibitor of CYP17A1) are effective treatments to improve the care for patients with metastatic prostate cancer. Despite initial favorable responses, a majority of patients ultimately develop into castration-resistant prostate cancer (CRPC); the duration of response for the most responding patients that have relapsed is within 1-2 years while that for the advanced cases (post chemotherapy) is even modest (4-6 months) (Kim, W., and Ryan, C. J. (2012) Androgen receptor directed therapies in castration-resistant metastatic prostate cancer. Curr Treat Options Oncol 13, 189-200).

Accumulating evidence suggests that advanced CRPC sustains active androgen receptor (AR) signaling for cancer cell growth despite the effective AR blockade (Yuan, X., Cai, C., Chen, S., Chen, S., Yu, Z., and Balk, S. P. (2014) Androgen receptor functions in castration-resistant prostate cancer and mechanisms of resistance to new agents targeting the androgen axis. Oncogene 33, 2815-2825). Potential resistance mechanisms that confer ligand-independent AR transactivation include mutations and alternative splicing of AR that generate constitutively active variants or associated protein that can enhance AR activity at very low levels of androgen and upregulation of CYP17A1 that increases the intratumoral androgen synthesis. In the majority of cases, AR remains active, despite the presence of low level of androgen. An unmet need at present is therefore to develop therapeutic modality which can diminish or eliminate the aberrantly activated AR activity.

From the pharmacological point of view, targeting a newly identified class of the epigenetic coactivator for AR and estrogen receptor to suppress their constitutive activity, such as the human histone demethylases of the KDM4/JMJD2 family (KDM4A-KDM4D) that erase a repressive chromatin mark K9me3/me2 is of therapeutic value (Berry, W. L., and Janknecht, R. (2013) KDM4/JMJD2 Histone Demethylases: Epigenetic Regulators in Cancer Cells. Cancer Res 73, 2936-2942; Wang, L., Chang, J., Varghese, D., Dellinger, M., Kumar, S., Best, A. M., Ruiz, J., Bruick, R., Pena-Llopis, S., Xu, J., Babinski, D. J., Frantz, D. E., Brekken, R. A., Quinn, A. M., Simeonov, A., Easmon, J., and Martinez, E. D. (2013) A small molecule modulates Jumonji histone demethylase activity and selectively inhibits cancer growth. Nature communications 4, 2035). Importantly, members of this family associate with AR and activate AR signaling (Wissmann, M., Yin, N., Muller, J. M., Greschik, H., Fodor, B. D., Jenuwein, T., Vogler, C., Schneider, R., Gunther, T., Buettner, R., Metzger, E., and Schule, R. (2007) Cooperative demethylation by JMJD2C and LSD1 promotes androgen receptor-dependent gene expression. Nat Cell Biol 9, 347-353; Shin, S., and Janknecht, R. (2007) Activation of androgen receptor by histone demethylases JMJD2A and JMJD2D. Biochem Biophys Res Commun 359, 742-746; Coffey, K., Rogerson, L., Ryan-Munden, C., Alkharaif, D., Stockley, J., Heer, R., Sahadevan, K., O'Neill, D., Jones, D., Darby, S., Staller, P., Mantilla, A., Gaughan, L., and Robson, C. N. (2013) The lysine demethylase, KDM4B, is a key molecule in androgen receptor signalling and turnover. Nucleic acids research 41, 4433-4446). Furthermore, KDM4A and KDM4B are highly expressed in prostate cancer and a progression factor for metastatic prostate cancer (Chu, C. H., Wang, L. Y., Hsu, K. C., Chen, C. C., Cheng, H. H., Wang, S. M., Wu, C. M., Chen, T. J., Li, L. T., Liu, R., Hung, C. L., Yang, J. M., Kung, H. J., and Wang, W. C. (2014) KDM4B as a target for prostate cancer: structural analysis and selective inhibition by a novel inhibitor. Journal of medicinal chemistry 57, 5975-5985). Additionally, KDM4B is hypoxia inducible, mimicking castration resistance, suggesting that KDM4B as a potential new target against CRPC. Two types of KDM4 inhibitors have been developed (Labbé, R. M., Holowatyj, A., and Yang, Z.-Q. (2014) Histone lysine demethylase (KDM) subfamily 4: structures, functions and therapeutic potential. American Journal of Translational Research 6, 1-15): a-ketoglutarate analogues and catalytic-site inhibitors. Of α-ketoglutarate analogues (N-oxalylglycines, hydroxamate analogues, pyridine 2,4-dicarboxylic acids, and 8-hydroxyquinolines), the inhibitor ML324 based on the 8-hydroxyquinoline skeleton shows submicromolar inhibitory activity toward JMJD2E/KDM4E (in vitro), good cell permeability and in vitro ADME properties (King, O. N., Li, X. S., Sakurai, M., Kawamura, A., Rose, N. R., Ng, S. S., Quinn, A. M., Rai, G., Mott, B. T., Beswick, P., Klose, R. J., Oppermann, U., Jadhav, A., Heightman, T. D., Maloney, D. J., Schofield, C. J., and Simeonov, A. (2010) Quantitative high-throughput screening identifies 8-hydroxyquinolines as cell-active histone demethylase inhibitors. PloS one 5, e15535; Rai, G., Kawamura, A., Tumber, A., Liang, Y., Vogel, J. L., Arbuckle, J. H., Rose, N. R., Dexheimer, T. S., Foley, T. L., King, O. N., Quinn, A., Mott, B. T., Schofield, C. J., Oppermann, U., Jadhav, A., Simeonov, A., Kristie, T. M., and Maloney, D. J. (2010) Discovery of ML324, a JMJD2 demethylase inhibitor with demonstrated antiviral activity. in Probe Reports from the NIH Molecular Libraries Program, Bethesda (Md.). pp). Liang et al. found that M324 blocked the KDM4A-dependent herpes virus-mediated replication in infected cells, which displayed promising therapeutic effect by targeting KDM4. Recently, potent catalytic-site inhibitors have been developed (Compound 54j, KDM4A/B, IC50=0.080 and 0.017 μM; Compound 35, 43 nM) (Bavetsias, V, Lanigan, R. M., Ruda, G. F., Atrash, B., McLaughlin, M. G., Tumber, A., Mok, N. Y., Le Bihan, Y. V, Dempster, S., Boxall, K. J., Jeganathan, F., Hatch, S. B., Savitsky, P., Velupillai, S., Krojer, T., England, K. S., Sejberg, J., Thai, C., Donovan, A., Pal, A., Scozzafava, G., Bennett, J. M., Kawamura, A., Johansson, C., Szykowska, A., Gileadi, C., Burgess-Brown, N. A., von Delft, F., Oppermann, U., Walters, Z., Shipley, J., Raynaud, F. I., Westaway, S. M., Prinjha, R. K., Fedorov, O., Burke, R., Schofield, C. J., Westwood, I. M., Bountra, C., Muller, S., van Montfort, R. L., Brennan, P. E., and Blagg, J. (2016) 8-Substituted Pyrido[3,4-d]pyrimidin-4(3H)-one Derivatives As Potent, Cell Permeable, KDM4 (JMJD2) and KDM5 (JARID1) Histone Lysine Demethylase Inhibitors. J Med Chem 59, 1388-1409; Korczynska, M., Le, D. D., Younger, N., Gregori-Puigjane, E., Tumber, A., Krojer, T., Velupillai, S., Gileadi, C., Nowak, R. P., Iwasa, E., Pollock, S. B., Ortiz Tones, I., Oppermann, U., Shoichet, B. K., and Fujimori, D. G. (2016) Docking and Linking of Fragments To Discover Jumonji Histone Demethylase Inhibitors. J Med Chem 59, 1580-1598). However, their drug-preferred features including cell permeability, efficacy, the selectivity and the in vivo evaluation have not been revealed.

Accumulating evidence shows that overexpression of KDM4A, KDM4B, and KDM4C is indicated in the efficient growth of human malignancies including breast, colorectal, lung, prostate, gastric, neuroblastoma, and ovarian cancers (Berry, W. L., and Janknecht, R. (2013) KDM4/JMJD2 histone demethylases: epigenetic regulators in cancer cells. Cancer Res 73, 2936-2942; Black, J. C., Manning, A. L., Van Rechem, C., Kim, J., Ladd, B., Cho, J., Pineda, C. M., Murphy, N., Daniels, D. L., Montagna, C., Lewis, P. W., Glass, K., Allis, C. D., Dyson, N. J., Getz, G., and Whetstine, J. R. (2013) KDM4A lysine demethylase induces site-specific copy gain and rereplication of regions amplified in tumors. Cell 154, 541-555; Yang, J., AlTahan, A. M., Hu, D., Wang, Y., Cheng, P. H., Morton, C. L., Qu, C., Nathwani, A. C., Shohet, J. M., Fotsis, T., Koster, J., Versteeg, R., Okada, H., Harris, A. L., and Davidoff, A. M. (2015) The role of histone demethylase KDM4.13 in Myc signaling in neuroblastoma. J Natl Cancer Inst 107, djv080; Han, Ren, J., Zhang, J., Sun, Y., Ma, F., Liu, Z., Yu. H., Jia, J., and Li, W. (2016) JMJD2B is required for Helicobacter pylori-induced gastric carcinogenesis via regulating COX-2 expression. Oncotarget 7, 38626-38637; Wilson, C., Chiu, L., Hong, Y, Karnik, T., Tadros, G., Mau, B., Ma, T., Mu, Y., New, J., Louie, R. J., Gunewardena, S., Godwin, A. K., Tawfik, O. W., Chien, J., Roby, K. F., and Krieg, A. J. (2017) The histone demethylase KDM4B regulates peritoneal seeding of ovarian cancer. Oncogene 36, 2565-2576). KDM4A regulates chromatin during DNA replication and stem cell genome reprogramming (Chung, Y G., Matoba, S., Liu, Eum, J. H., Lu, F., Jiang, W., Lee, J. E., Sepilian, V, Cha. Y., Lee, D. R., and Zhang, Y. (2015) Histone Demethylase Expression Enhances Human Somatic Cell Nuclear Transfer Efficiency and Promotes Derivation of Pluripotent Stern Cells. Cell Stem Cell 17, 758-766). KDM4B controls DNA repair, mitochondrial apoptosis, and reprograms the genome of somatic cell cloned embryo to conquer its arrest (Young, L. McDonald, W., and Hendzel, M. J. (2013) Kdm4b histone demethylase is a DNA damage response protein and confers a survival advantage following gamma-irradiation. J Biol Chem 288, 21376-21388; Li, E L, Yang, X., Wang, G., Li, X., Tao, D., Flu, J., and Luo, X. (2016) KDM4B plays an important role in mitochondrial apoptosis by upregulating HAX1 expression in colorectal cancer. Oncotarget 7, 57866-57877; Liu, W, Liu, X., Tang, C., Gao, Y., Gao, R., Kou, X., Zhao, Y., Li, J., Wu, Y., Xiu, W., Wang, S., Yin, J., Liu, W., Cai, T., Wang, H., Zhang, Y., and Gao, S. (2016) Identification of key factors conquering developmental arrest of somatic cell cloned embryos by combining embryo biopsy and single-cell sequencing. Cell Discov 2, 16010). KDM4A can also associate with co-repressor NCoR to suppress the TRAIL-DR5 pathway (Wang, J., Wang, L. Y, Cai, D., Duan, Z., Zhang, Y, Chen, P., Zou, J. X., Xu, J., Chen, X., Kung, H. J., and Chen, H. W. (2016) Silencing the epigenetic silencer KDM4A for TRAIL and DR5 simultaneous induction and antitumor therapy. Cell Death Differ 23, 1886-1896) and function as a key regulator of tumor metabolism through E2F1 (Wang, L. Y, Hung, C. L., Chen, Y. R., Yang, J. C., Wang, J., Campbell, M., Izumiya, Y., Chen, H. W., Wang, W. C., Ann, D. K., and Kung, H. J. (2016) KDM4A Coactivates E2F1 to Regulate the PDK-Dependent Metabolic Switch between Mitochondrial Oxidation and Glycolysis. Cell Rep 16, 3016-3027). KDM4A, KDM4B, and KDM4C act as coactivators of androgen receptor and estrogen receptor, which are considered as promising epigenetic drug targets (Shin, S., and Janknecht, R. (2007) Activation of androgen receptor by histone demethylases JMJD2A and JMJD2D. Biochem Biophys Res Commun 359, 742-746; Shi, L., Sun, L., Li, Q., Liang, J., Yu, W., Yi, X., Yang, X., Li, Y., Han, X., Zhang, Y, Xuan, C., Yao, Z., and Shang, Y. (2011) Histone demethylase JMJD2B coordinates H3K4/H3K9 methylation and promotes hormonally responsive breast carcinogenesis. Proc Natl Acad Sci USA 108, 7541-7546; Berry, W. L., Shin, Lightfoot, S. A., and Janknecht, R. (2012) Oncogenic features of the JMJD2A histone demethylase in breast cancer, Int J Oncol 41, 1701-1706; Coffey, K., Rogerson, L., Ryan-Munden, C., Alkharaif, D., Stockley, J., Heer, R., Sahadevan, K., O'Neill, D., Jones, D., Darby, S., Staller, P., Mantilla, A., Gaughan, L., and Robson, C. N. (2013) The lysine demethylase, KDM4B, is a key molecule in androgen receptor signalling and turnover. Nucleic Acids Res 41, 4433-4446; Chu, C. H., Wang, L. Y., Hsu, K. C., Chen, C. C., Cheng, H. H., Wang, S. M., Wu, C. M., Chen, T. J., Li, L. T., Liu; R., Hung, C. L., Yang, J. M., Kung, H. J.; and Wang, W. C. (2014) KDM4B as a target for prostate cancer: structural analysis and selective inhibition by a novel inhibitor. J Med Chem 57, 5975-5985),

### BRIEF DESCRIPTION OF THE DRAWINGS

FIG. 1A: Scheme of KDM4B inhibitor discovery over the library of natural products. iGEMDOCK was used to generate docked poses of KDM4B by screening the compound library TCM Database@Taiwan (http://tem.emu.edu.tw).

FIG. 1B: Residual KDM4B enzymatic activity of available top-ranking compounds.

FIG. 2A: Inhibition kinetics of Myricetin against KDM4A/KDM4B/KDM4C. The inset in each panel shows the double-reciprocal form, where the 1/relative activity is plotted versus 1/[H3K9me3] at various fixed concentrations of Myricetin as indicated.

FIG. 2B: Inhibition of H3 demethylation by bacteria-expressed KDM4A, KDM4B, or KDM4C in the presence of Myricetin by western blot analysis. The reaction mixture containing 10 μM enzyme, 100 μM inhibitor or blank buffer, and 5 μM of H3 in 50 mM HEPES, pH 7.5, 1 mM AKG, 2 mM ascorbate, and 50 μM Fe(II) is incubated at 37° C. for 30 min, followed by western blot analysis. H3 lysine modifications are probed with H3K9me3 and H3K36me3 antisera, respectively.

FIG. 3A: Expression of KDM4B in normal immortalized RWPE-1 and PZ-HPV-7 cells, and prostate cancer cell lines LNCaP, C4-2, C4-2B, CWR22Rv1, PC3 and DU-145. AS, androgen sensitive; AR+, androgen receptor-positive; AR-, androgen receptor-negative; CRPC, castration-resistant prostate cancer.

FIG. 3B: Generation of KDM4B-knockdown LNCaP, C4-2B, and DR-145 cells based on the lentivirus approach.

FIG. 3C: MTT cell proliferation assay (upper panel) and cell number counting (lower panel) of control (LKO) and KDM4B-knockdown (sh4B#1 and sh4B#2) prostate cancer cells at indicated time points. Data presented are means±SD from three independent experiments.

FIG. 4: C4-2B xenograft tumor growth is impaired for the KDM4B-knockdown line. Data are presented the growth of each tumor via the time trend. The p-value to test the KDM4B knockdown effect on tumor size growth over time is 0.0013. The tumor size difference between two groups at a given time point is 3.13 mm in average (95% Cl: −132.08, 138.34).

FIG. 5A: Myricetin exhibits cell cytotoxicity against androgen-dependent and -independent prostate cancer cells. Cells (normal immortalized PZ-HPV-7, androgen-dependent LNCaP, and androgen-independent C4-2B) are treated with different concentrations of Myricetin over 4 days as indicated, followed by MTT assay.

FIG. 5B: H3K9me3 and H3K36me3 levels in Myricetin-treated C4-2B cells for two days. The H3K9me3 and H3K36me3 signals are detected in cell lysates by western blot analysis.

FIG. 6: (A) UV spectra of Myricetin revealed an absorbance peak at 385 nm. (B) A linear relationship between absorbance and the concentration of Myricetin. (C-E) Comparison of cell viability after C4-2B cells treated with PLGA-encapsulated Myricetin (PLGA-Myricetin) versus free Myricetin at different concentrations as indicated. IC50 of free Myricetin=38.3 μM (D). IC50 of PLGA-Myricetin=24.3 μM (E).

FIG. 7A: Myricetin impairs tumor growth in C4-28 xenografts. 1×10⁶ C4-2B cells are implanted subcutaneously into hindlimb of nude mice. PLGA-Myricetin (intraperitoneal injected, 20 mg/Kg, three times a week) and enzalutamide (oral gavage feeding, 12.5 mg/Kg, five times a week) are administered after 14 days implantation, tumor volumes are measured for up to 3 weeks. Tumor volume is calculated using the formula (volume=length×width×height×0.52). Tumor size growth is observed over time. V, vehicle; Myricetin, PLGA-Myricetin; Enza, enzalutamide; Enza+ Myricetin, the combined enzalutamide plus Myricetin.

FIG. 7B: Ki-67 and CD31 immunohistochemistry staining of the tumor sections isolated from the xenografts.

FIG. 8A: Inhibitory effect for Myricetin administered alone on C4-2B cells.

FIG. 8B: Inhibitory effect for enzalutamide administered alone on C4-2B cells.

FIG. 8C: The cytotoxic effects of Myricetin and enzalutamide used alone or in combination on a prostate cancer cell line.

FIG. 8D: Combination Index (CI) for different concentrations of Myricetin and enzalutamide administered to prostate cancer cells.

### SUMMARY OF THE INVENTION

The present invention relates to a method for inhibiting activity of a protein selected from the group consisting of KDM4A, KDM4B, and KDM4C, comprising the step of exposing the protein to an effective inhibiting amount of a compound having the formula

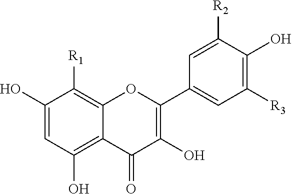

wherein

R₁ is H or C₇O₅H₈;

R₂ is H, OH, or OCH₃; and

R₃ is H or OH.

The present invention also relates to a method for treating or lessening the severity of a disease, condition, or disorder in which modulation of KDM4A, KDM4B, or KDM4C is beneficial, which method comprises administering to a subject suffering from said disease, condition, or disorder a therapeutically-effective amount of:

a) a compound having the formula

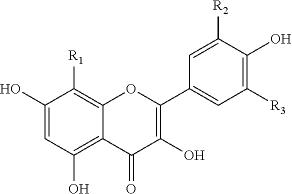

wherein
R₁ is H or C₇O₅H₈;
R₂ is H, OH, or OCH₃; and
R₃ is H or OH; or

b) a composition comprising the compound and a pharmaceutically acceptable adjuvant, vehicle, or carrier.

### DETAILED DESCRIPTION OF THE INVENTION

The present invention is concerned with methods of inhibiting KDM4A/KDM4B/KDM4C, which include subjecting a cell to an effective concentration of a KDM4A/KDM4B/KDM4C inhibitor such as one of the compounds disclosed herein. It is believed that the use of such inhibitors will serve to treat cancer in conjunction with other anti-cancer agents, chemotherapy, resection, radiation therapy, and the like. The compounds of this invention, besides acting as KDM4A/KDM4B/KDM4C inhibitors, may have other effects that can lead to antitumor activity.

This study screens for natural compounds that blocked the KDM4B activity via the structure-based approach over the natural compound library TCM Database@Taiwan (Chen, C. Y. (2011) TCM Database@Taiwan: the world's largest traditional Chinese medicine database for drug screening in silico. PloS one 6, e15939) (http://tcm.cmu.edu.tw). The natural compound Myricetin that exhibits a competitive inhibition mode against KDM4B is identified. Myricetin also shows inhibition against other members of the KDM4 family. A liposome-encapsulated Myricetin form is also employed and demonstrates a higher level of cell cytotoxicity both for AR+, androgen-sensitive (LNCaP) and AR+, castration-resistant (CWR22Rv1 and C4-2B) cells. By the use of the xenograft models, it is shown that knockdown of KDM4B significantly impairs the tumor growth. Pharmacological inhibition by Myricetin, enzalutamide which is an FDA-approved drug to treat metastatic prostate cancer, and the combined treatment of Myricetin and enzalutamide for C4-2B xenografts in nude mice is tested. The results show that Myricetin, enzalutamide, and the combined treatment have significant antitumor activity compared with the vehicle control group in the C4-2B xenograft model. Combining Myricetin with enzalutamide, the treatment also reduces the rate of tumor growth, comparing to the treatment of enzalutamide alone. In sum, this xenograft study proves that the combination of Myricetin (or liposome-encapsulated Myricetin) with enzalutamide is an effective treatment for castration resistant prostate cancer.

This study also demonstrates that Myricetin analogues (such as Isorhamnetin, Gossypin, Quercetin, and Kaempferol) have similar inhibitory effects against KDM4B activity.

Therefore, the present invention provides a compound having the formula

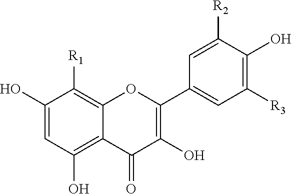

or a pharmaceutically acceptable salt thereof, wherein:

R₁ is H or C₇O₅H₈;

R₂ is H, OH, or OCH₃; and

R₃ is H or OH.

The present invention also provides a method for inhibiting activity of a protein selected from the group consisting of KDM4A, KDM4B, and KDM4C, comprising the step of exposing the protein to an effective inhibiting amount of a compound having the formula

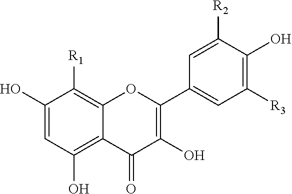

wherein

R₁ is H or C₇O₅H₈;

R₂ is H, OH, or OCH₃; and

R₃ is H or OH.

In an embodiment, the compound is selected from Myricetin, Quercetin, Kaempferol, Gossypin, or Isorhamnetin. In another embodiment, the compound is liposome-encapsulated.

The present invention further provides a method for treating or lessening the severity of a disease, condition, or disorder in which modulation of KDM4A, KDM4B, or KDM4C is beneficial, which method comprises administering to a subject suffering from said disease, condition, or disorder a therapeutically-effective amount of:

a) a compound having the formula

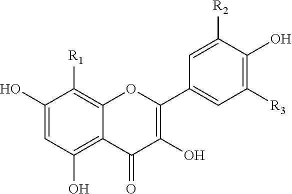

wherein

R₁ is H or C₇O₅H₈;

R₂ is H, OH, or OCH₃; and

R₃ is H or OH; or

b) a composition comprising the compound and a pharmaceutically acceptable adjuvant, vehicle, or carrier.

In an embodiment, the disease, condition, or disorder is a cancer selected from breast cancer, colorectal cancer, lung cancer, prostate cancer, gastric cancer, neuroblastoma cancer, or ovarian cancer. In another embodiment, the prostate cancer is castration resistant prostate cancer.

In an embodiment, the compound is further combined with enzalutamide as a combination therapy. Preferably, the concentration ratio of the compound and enzalutamide in the combination is from about 1:0.4 to about 1:0.7.

In an embodiment, the compound is selected from Myricetin, Quercetin, Kaempferol, Gossypin, or Isorhamnetin. In another embodiment, the compound is liposome-encapsulated.

### EXAMPLES

The examples below are non-limiting and are merely representative of various aspects and features of the present invention.

### Example 1: Identifying an Inhibitor Towards KDM4A, KDM4B, and KDM4C Via a Structure-Guided Approach

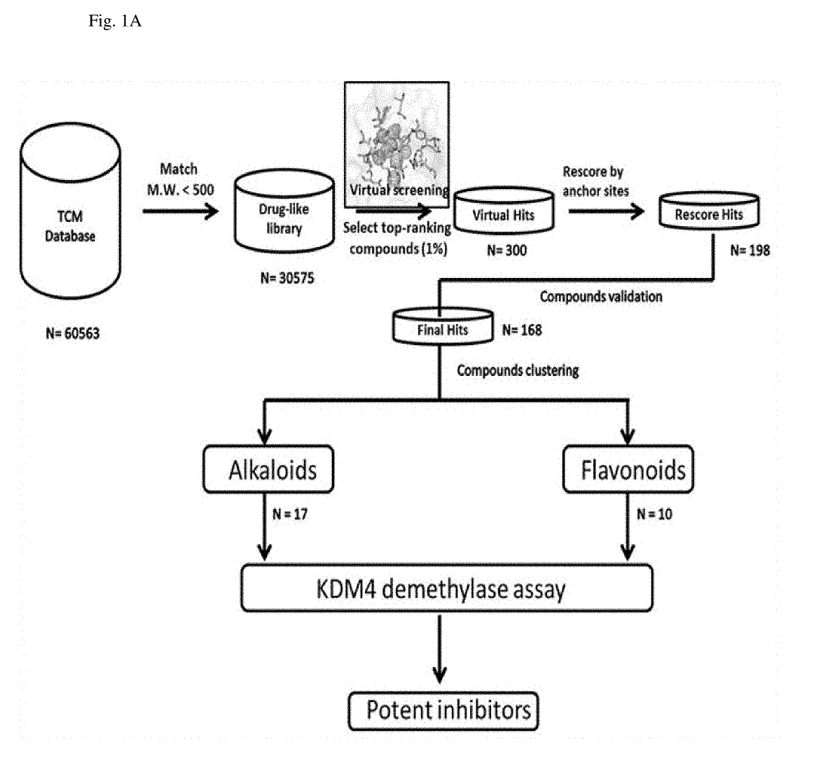

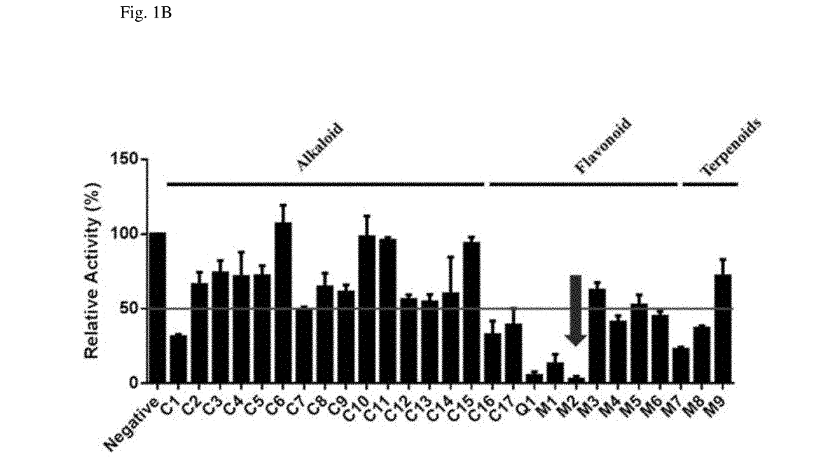

A graphical-automatic drug design system, iGEMDOCK, was utilized to screen for natural compounds against KDM4B over Traditional Chinese Medicine Database @ Taiwan (<500 Daltons, n=30575) {Lipinski, 2004 #7}. SiMMap was further utilized to rescore top-ranking 300 interaction energy compounds. Of those, the top-ranking compounds (n=198) were classified based on four groups of natural products: 94 compounds were alkaloid, 12 compounds were terpenoid, 62 compounds were flavonoid and 30 compounds were polyketide and organic acid. Fourteen alkaloid compounds, ten flavonoid compounds, and three terpenoid compounds that were commercially available and possessed stability were characterized (FIG. 1A). Of those, a flavonoid M2 (i.e. Myricetin) had the highest inhibitory effect against KDM4B (FIG. 1B).

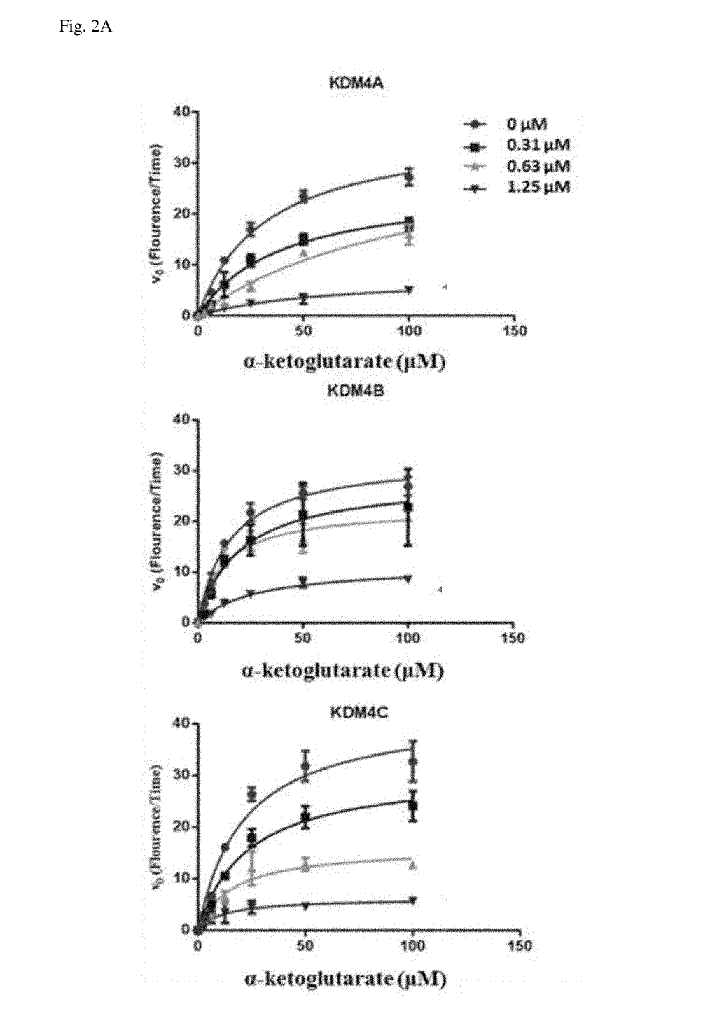

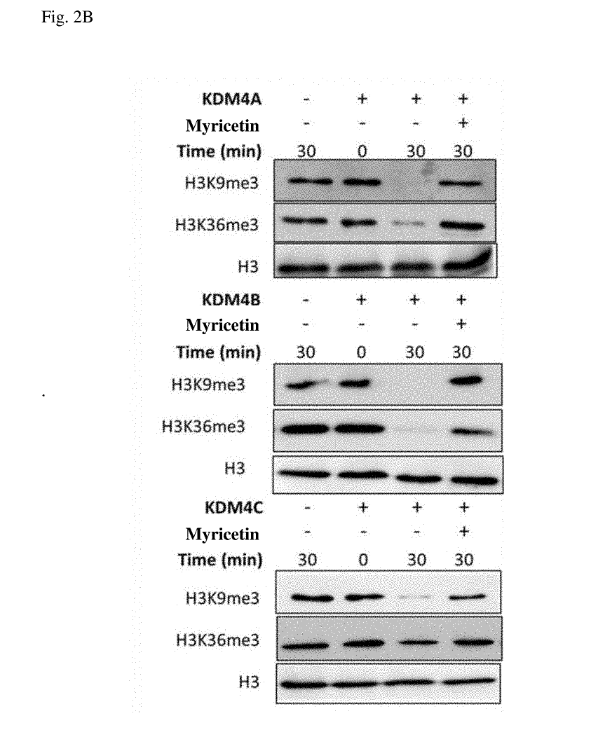

Myricetin displayed inhibitory activity toward KDM4A, KDM4B, and KDM4C respectively (IC50: 1.13 μM for KDM4A, 1.30 μM for KDM4B and 1.12 μM for KDM4C). Enzymatic inhibitory kinetics displayed a competitive mode toward α-ketoglutarate (FIG. 2A).

It was next tested whether Myricetin specifically blocked the demethylation activity by KDM4 members using H3 as the substrate and probed for H3K9me3 and H3K36me3 in the presence of KDM4B or KDM4C. As shown in FIG. 2B, western blotting analysis showed that the signal of H3K9me3 was greatly reduced in the presence of KDM4B or KDM4C for 30 min compared with that in the controls. Addition of Myricetin significantly reduced such an activity. No difference was found for H3K27me3 in the presence of KDM4B. These results demonstrated that KDM4B and KDM4C were specifically blocked by Myricetin.

Inhibitory effects of Myricetin analogues (such as Isorhamnetin, Gossypin, Quercetin, and Kaempferol) against KDM4B activity were also tested. The results were shown in Table 1. The results showed that all of these Myricetin analogues have significant inhibitory effects against KDM4B. Given the structural similarity between KDM4B and KDM4A/KDM4C, these compounds were considered have inhibitory effects against KDM4A/KDM4C.

### Example 2: Depletion of KDM4B Inhibited the Growth of Androgen Receptor-Positive (AR+) Prostate Cancer Cells

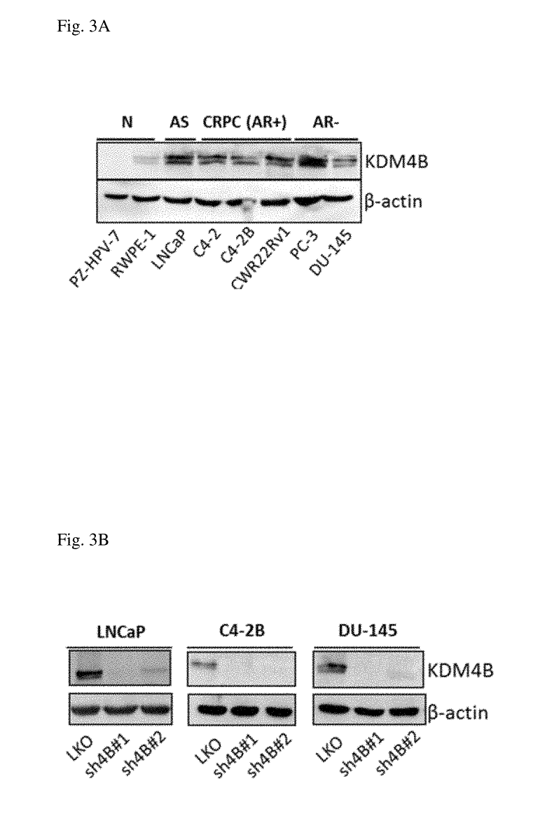

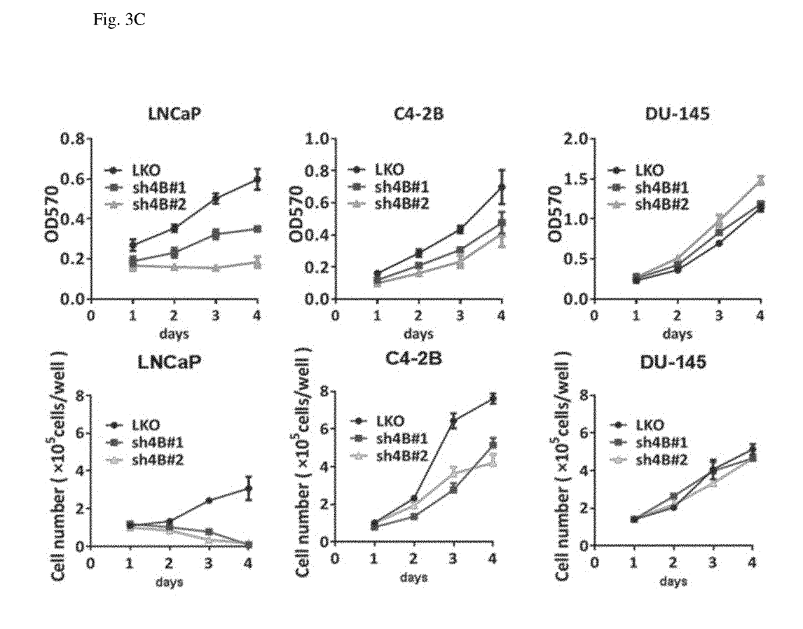

*: 100 μM compounds ocked cell growth of prostate cancer cells, LNCaP (AR+, androgen-dependent), C4-2, C4-2B, and CWR22Rv1 (AR+, androgen-independent) plus RWPE-1 and PZ-HPV-7 (immortalized normal epithelial cells) were characterized. FIG. 3A showed that the expression of KDM4B was much higher in all prostate cancer cells than that in immortalized normal lines. By the use of lentiviral-based approach to deplete KDM4B, knocking down the expression of KDM4B for AR+LNCaP and C4-2B significantly reduced the cell growth (FIG. 3B and FIG. 3C). Conversely, there was essentially no effect for AR-negative DU145 cells (FIG. 3B and FIG. 3C).

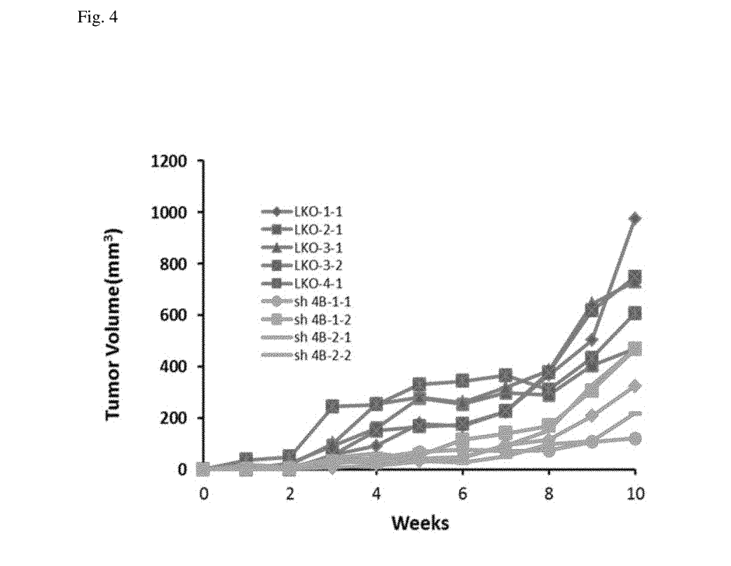

Whether KDM4B was required for androgen-independent growth was next characterized using an animal model. This was done by utilizing CRPC line C4-2B predisposed to growing as tumors in nude mice. Mice were implanted with the control (LKO) or KDM4B-knockdown C4-2B cells (sh4B). FIG. 4 showed that KDM4B-knockdown expression in C4-2B xenografts exhibited significant diminution in tumor growth via the time trend (p=0.0013). Furthermore, results from knockdown of KDM4A (project 1) and KDM4C (project 3) also showed the similar trend. Together, the results strongly supported the proposal that targeting KDM4 as a new therapeutic strategy against CRPC.

### Example 3: Myricetin Inhibited the Growth of C4-2B Cells and Blocked the Demethylation of H3K9me3

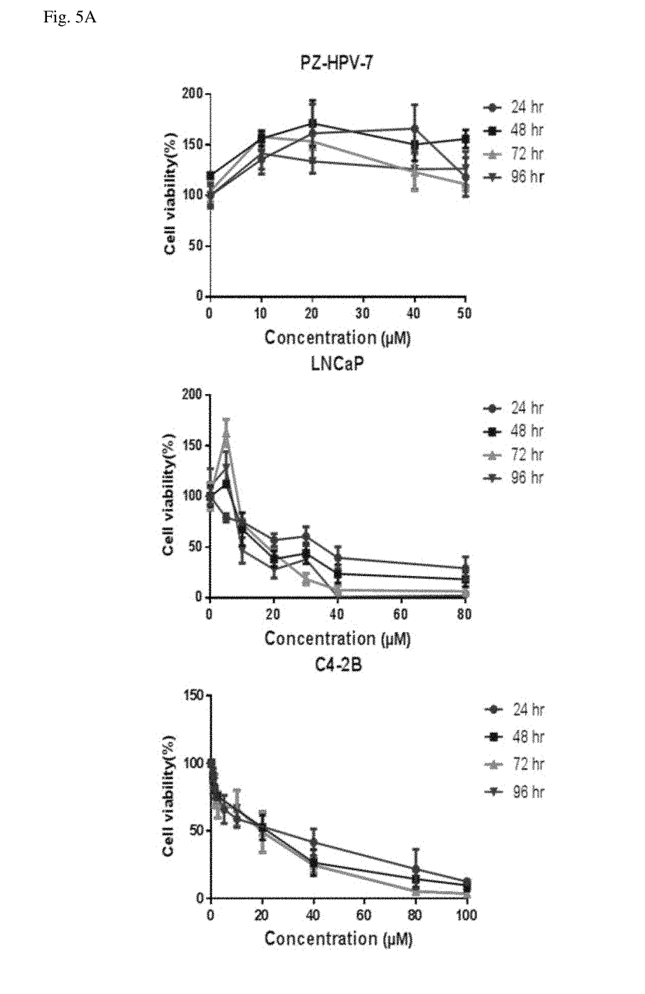

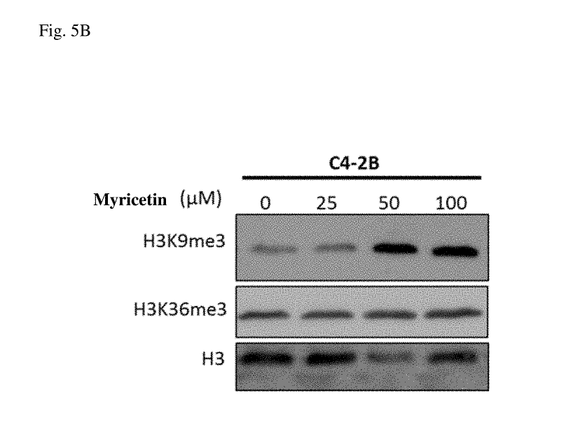

Whether Myricetin blocked cell growth of AR+ cells was next evaluated. FIG. 5A showed that a significantly reduced level of cell growth in Myricetin-treated LNCaP via a dose-dependent manner. Cell proliferation of C4-2B was also blocked by Myricetin. In contrast, there was essentially no effect for the immortalized normal PZ-HPV-7 line. A dose-dependent increased level of H3K9me3 in Myricetin-treated C4-2B was observed in Myricetin-treated C4-2B cells as compared with a relatively comparable signal of H3K36me3, thus suggesting that Myricetin specifically blocked the demethylation of H3K9me3 in C4-2B cells and reduced the cell proliferation (FIG. 5B).

### Example 4: PLGA-Myricetin Impaired the Tumor Growth of C4-2B Xenografts and the Combined PLGA-M2 Plus Enzalutamide Treatment Improved the Efficacy of Enzalutamide Alone

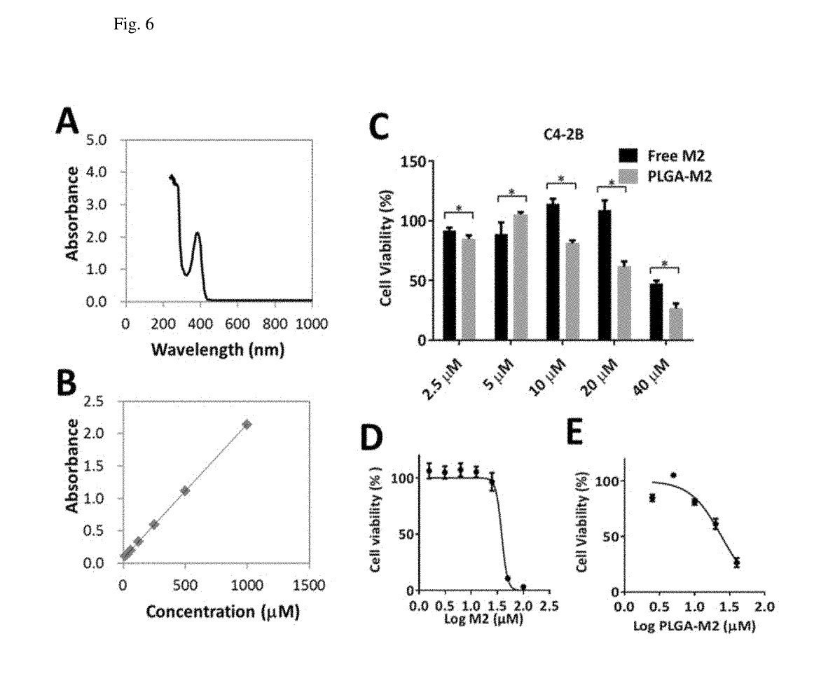

To test whether Myricetin could reduce the tumor growth in vivo, Myricetin was also formulated as a liposome-encapsulated form to increase its half-life in plasma in collaboration with Dr. Chen (Barve, A., Chen, C., Hebbar, V, Desiderio, J., Saw, C. L., and Kong, A. N. (2009) Metabolism, oral bioavailability and pharmacokinetics of chemopreventive kaempferol in rats. Biopharm Drug Dispos 30, 356-365). FIG. 6 showed that Myricetin was effectively prepared as the PLGA-encapsulated Myricetin form (PLGA-Myricetin) (FIG. 6, (A) and (B)). PLGA-Myricetin displayed a stronger cytotoxicity than did the free Myricetin form (IC50, 24.3 μM vs. 38.3 μM) (FIG. 6, (C)-(E)).

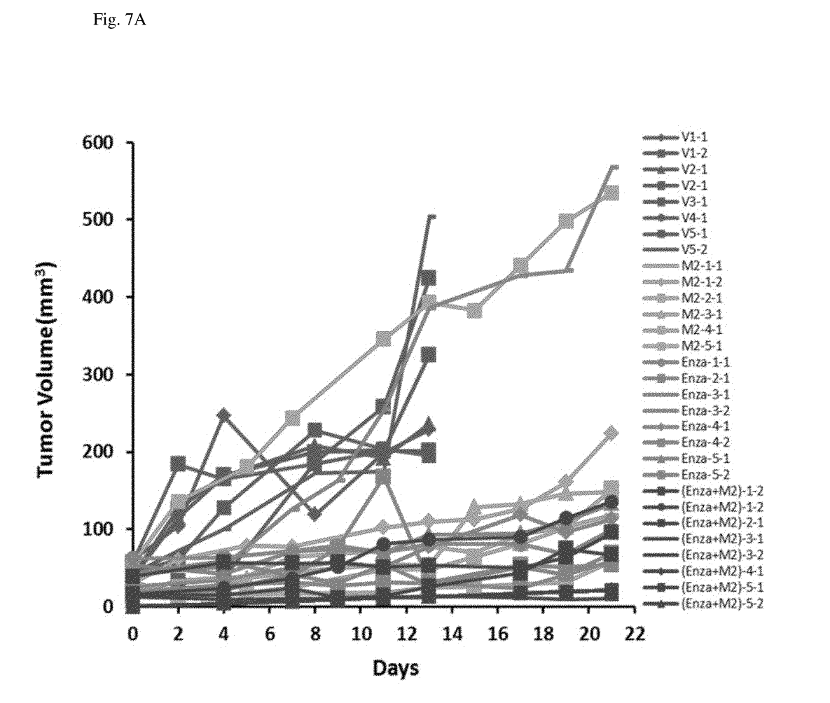

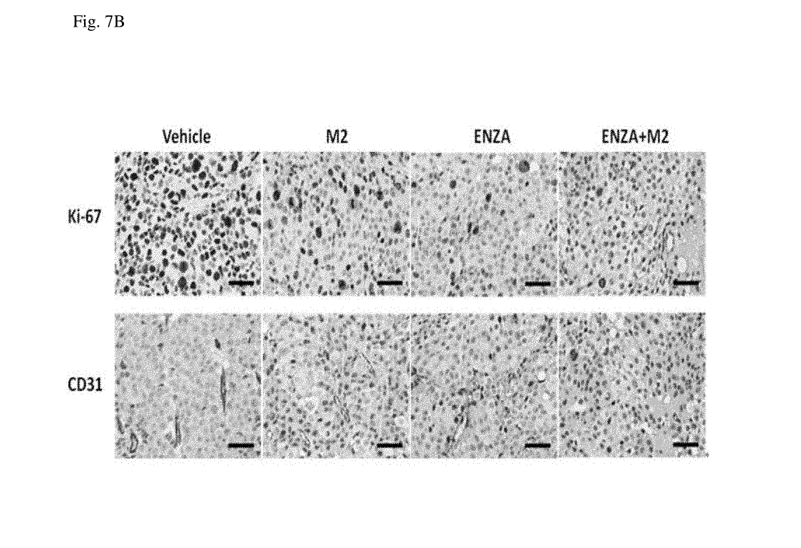

To assess the efficacy of PLGA-Myricetin in vivo, xenografts generated from C4-2B cells were treated with vehicle, Myricetin, enzalutamide which was an FDA-approved drug to treat metastatic prostate cancer, and the combination of Myricetin and enzalutamide. To compare the efficacy of Myricetin vs. vehicle over time using data from 0 to 13 days, under the same baseline value at a given time point, the tumor size for the Myricetin group was 46.81 mm smaller averagely than it in vehicle group (95% CI: −124.37, 30.78, Myricetin minus vehicle) with p-value 0.0002. A comparison between enzalutamide vs. the combined treatment of iMyricetin and enzalutamide using data from day 0 to day 21 had the estimation that the tumor size using the combined treatment group was 4.24 mm smaller in average than in enzalutamide group (95% CI: −72.46, 80.65) with p-value 0.056. Analogously, when comparing the treatment effect of Myricetin vs. the combination treatment of Myricetin plus enzalutamide using the complete data from day 0 to day 21, the tumor size using the combined treatment is −9.8 mm smaller in average than it in the Myricetin group (95% CI: −90.28, 71.40) with p-value less than 0.0001 (FIG. 7A).

Correspondingly, there was a significantly lower expression of proliferation marker Ki-67 in PLGA-Myricetin-treated xenografts and enzalutamide-treated xenografts. There was essentially no positive Ki-67 signal in xenografts for the combined treatment (FIG. 7B). The endothelial cell-specific marker CD31 in xenografts was also determined. A significantly higher level of CD31 was seen in the vehicle group as compared with PLGA-Myricetin-treated and enzalutamide-treated groups (FIG. 7B). Essentially no CD31 signal was detected in the combined treatment group. These results showed that PLGA-Myricetin was effective to block the C4-2B cell and xenograft models. Furthermore, the combined Myricetin-enzalutamide treatment provided a significantly improved effect to reduce the rate of tumor growth as compared to the treatment of enzalutamide alone.

### Example 5: KDM4B Regulated Tumor Metabolism Via Partnership with cMyc

Aberrant cellular metabolism of cancer cells was recognized as a hallmark of malignant transformation for its survival and proficient growth. Dominant aerobic glycolysis over mitochondria oxidative phosphorylation and dysregulated corticosteroid metabolism had been found in aggressive prostate cancer (Cutruzzolà, F., Giardina, G., Marani, M., Macone, A., Paiardini, A., Rinaldo, S., and Paone, A. (2017) Glucose Metabolism in the Progression of Prostate Cancer. Frontiers in Physiology 8; Li, J., Alyamani, M., Zhang, A., Chang, K. H., Berk, M., Li, Z., Zhu, Z., Petro, M., Magi-Galluzzi, C., Taplin, M. E., Garcia, J. A., Courtney, K., Klein, E. A., and Sharifi, N. (2017) Aberrant corticosteroid metabolism in tumor cells enables GR takeover in enzalutamide resistant prostate cancer. eLife 6). Analysis of the extracellular acidification rate (ECAR) revealed that knockdown of KDM4B in C4-2B displayed significantly reduced EACRs (data not shown). Correspondingly, metabolic genes of aerobic glycolysis (GAPDH, ALDOA, PGM1, ENO1, PKM2, LDHA) were down modulated. Since c-Myc was crucial to stimulate glycolysis including enolase and LDHA (Miller, D. M., Thomas, S. D., Islam, A., Muench, D., and Sedoris, K. (2012) c-Myc and cancer metabolism. Clin Cancer Res 18, 5546-5553), it was reasoned that KDM4B functioned as a coactivator of c-Myc. Knocking down the expression of KDM4B also significantly reduced the transactivation activity mediated by c-Myc. The results showed that KDM4B and cMyc associated with each other in an immunoprecipitation analysis in C4-2B (data not shown). Taken together, these data suggested that KDM4B was a major metabolic regulator in C4-2B.

### Example 6: The Synergistic Effect of Myricetin and Enzalutamide

In this example, the cytotoxic effects of Myricetin and enzalutamide used alone or in combination on a prostate cancer cell line were calculated according to Chou. et al. (T. C. Chou, “Theoretical Basis, Experimental Design, and Computerized Simulation of Synergism and Antagonism in Drug Combination Studies.” Pharmacol Rev 58:621-681, 2006) The Chou algorithm was employed to calculate the Combination Index (CI) which was shown in Table 2.

TABLE 2
Criteria for Combination Index (CI)

| | |
|---|---|
| CI ≤ 0.3 | Strong Synergy |
| 0.3 < CI ≤ 0.85 | Synergy |
| 0.85 < CI ≤ 1.2 | Additive |
| 1.2 < CI ≤ 3.3 | Antagonism |
| CI ≥ 3.3 | Strong |
| | Antagonism |

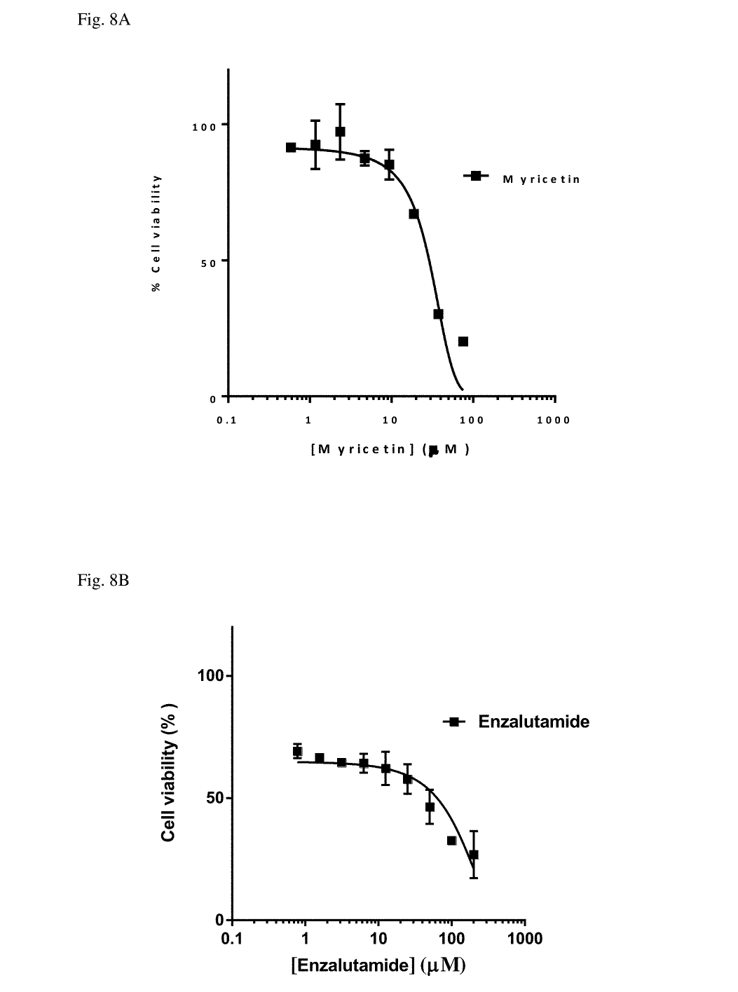

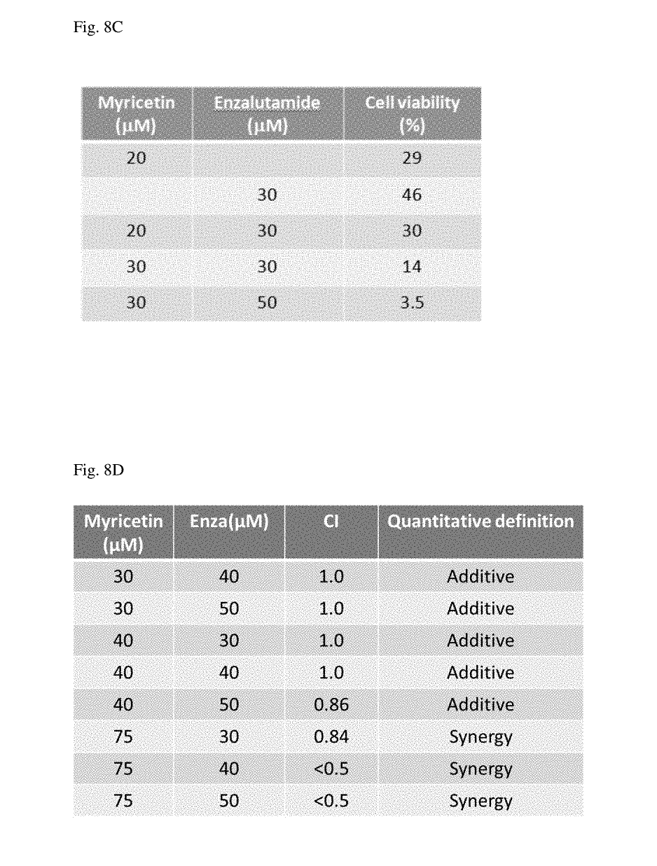

FIGS. 8A and 8B showed the inhibitory effects for Myricetin and enzalutamide administered alone on C4-2B cells When the Myricetin and enzalutamide were administered in combination, the cell viability was 30% (Myricetin 20 μM; enzalutamide 30 μM) and reduced to 14% by administering more Myricetin (Myricetin 30 μM; enzalutamide 30 μM) (FIG. 8C). In addition, Myricetin alone had better cytotoxic effect on the prostate cancer cells than enzalutamide alone (FIG. 8C).

Combinations of different concentrations of Myricetin and enzalutamide were administered to the prostate cancer cells and Combination Index (CI) was calculated based on the Chou-Talalay algorithm and shown in FIG. 8D. The result showed there was an addictive effect when the concentrations of Myricetin and enzalutamide were 30 μM and 40 μM, respectively. When the concentrations of Myricetin and enzalutamide were 75 μM and 30-50 μM, respectively (i.e., the concentration ratio of Myricetin:enzalutamide was from about 1:0.4 to about 1:0.7), the cytotoxic effects were synergistic.

One skilled in the art readily appreciates that the present invention is well adapted to carry out the objects and obtain the ends and advantages mentioned, as well as those inherent therein. The methods and uses thereof are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations, which are not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1-10. (canceled)

11. A method for treating or lessening the severity of a disease, condition, or disorder in which modulation of KDM4A, KDM4B, or KDM4C is beneficial, which method comprises administering to a subject suffering from said disease, condition, or disorder a therapeutically-effective amount of:
a) a combination of enzalutamide and a compound having the following formula 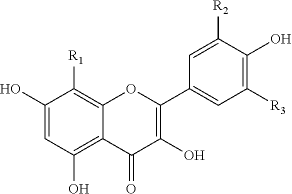
wherein
R₁ is H or C₇O₅H₈;
R₂ is H, OH, or OCH₃; and
R₃ is H or OH;
wherein the concentration ratio of enzalutamide and the compound is from about 0.4:1 to about 0.7:1; or
b) a composition comprising the combination and a pharmaceutically acceptable adjuvant, vehicle, or carrier.

12. The method of claim 11, wherein the disease, condition, or disorder is a cancer selected from breast cancer, colorectal cancer, lung cancer, prostate cancer, gastric cancer, neuroblastoma cancer, or ovarian cancer.

13. The method of claim 12, wherein the prostate cancer is castration resistant prostate cancer.

14. (canceled)

15. (canceled)

16. The method of claim 11, wherein the compound is selected from Myricetin, Quercetin, Kaempferol, Gossypin, and Isorhamnetin.

17. The method of claim 11, wherein the compound is liposome-encapsulated.
